(19) United States
(12) Patent Application Publication
(10) Pub. No.: US 2016/0256086 A1
(43) Pub. Date: Sep. 8, 2016

(54) Non-Invasive, Bioelectric Lifestyle Management Device

(71) Applicant: Co-Optical, Pullman, WA (US)

(72) Inventors: Samuel Steven Byrd, Pullman, WA (US); Zane Alexander Duke, Pullman, WA (US); Amber DeAnn Graviet, Pullman, WA (US); AlQassem Omar Shaaban Naim, Pullman, WA (US)

(21) Appl. No.: 15/059,671

(22) Filed: Mar. 3, 2016

Related U.S. Application Data
(60) Provisional application No. 62/127,820, filed on Mar. 3, 2015.

(51) Int. Cl. A61B 5/145; A61B 5/0402; A61B 5/024; A61B 5/0488; A61B 5/0496; A61B 5/00; A61B 5/0476

(52) U.S. Cl. CPC A61B 5/14532; A61B 5/0059; A61B 5/0402; A61B 5/0476; A61B 5/0488; A61B 5/4845; A61B 5/0496; A61B 5/742; A61B 5/6803; A61B 5/7203; A61B 5/024; A61B 5/14546

(57) ABSTRACT

The techniques discussed herein ascertain a biological condition via bioelectric signals. In at least one example, the techniques capture bioelectric signals of an organism, isolate from the bioelectric signals of the organism the bioelectric signals emitted from an eye of an organism, and correlate properties of the bioelectric signal emitted from the eye with biological conditions of the organism such as, for example, blood glucose level, a heart rate, a blood ketone level, a blood alcohol content, a hydration level, a blood albumin level, and/or a blood electrolyte level

### PRIORITY

This application claims priority under 37 C.F.R 1.78 to Provisional Patent Application No. 62/127,820, filed Mar. 3, 2015 and titled, “Non-Invasive, Bioelectric Lifestyle Management Device.”

### BACKGROUND

Various medical conditions require regular monitoring of biological conditions such as blood glucose levels. Moreover, increased monitoring of biological conditions in otherwise healthy organisms can increase the chances of detecting abnormalities in organism health that can lead to earlier diagnoses and better prognoses for individual organisms. However, regular testing may be burdensome or left undone because the methods required to test biological conditions of an organism may be invasive, inconvenient for regular testing, or may require medical knowledge to perform. For example, previous solutions for monitoring blood glucose levels have required using a lancet to sample blood, which is invasive, and previous solutions to detect conditions such as retinopathy, neurological, and ophthalmological disorders have required the involvement of a medical professional.

### BRIEF DESCRIPTION OF THE DRAWINGS

The detailed description is described with reference to the accompanying figures. In the figures, the left-most digit(s) of a reference number identifies the figure in which the reference number first appears. The same reference numbers in different figures indicate similar or identical items.

FIG. 1 is a diagram depicting an example environment in which examples of non-invasive bioelectric lifestyle management can operate.

FIG. 2 is a block diagram depicting an example device that can implement non-invasive bioelectric lifestyle management, according to various examples.

FIG. 3 is an example wearable device for non-invasive bioelectric lifestyle management.

FIG. 4A is top view of an example wearable device for non-invasive bioelectric lifestyle management,

FIG. 4B is a user's view through an example wearable device for non-invasive bioelectric lifestyle management.

FIG. 5 is a flow diagram illustrating an example process to non-invasively manage a user's lifestyle using electrical signals.

FIG. 6 is a flow diagram illustrating an example process to non-invasively manage a user's lifestyle using bioelectric signals.

FIG. 7 is a flow diagram illustrating an example process to non-invasively manage a user's lifestyle using bioelectric.

### DETAILED DESCRIPTION

### Overview

This disclosure is directed to techniques to provide non-invasive bioelectric lifestyle management. As used herein, the term “bioelectric signals” is used to describe a bioelectric potential over time. It is contemplated that unless specifically stated in instances that the term “bioelectric potentials” is used, “bioelectric signals” could equally be used since, even if the bioelectric potential drops to zero, this is still the amplitude of that bioelectric signal at that point in time.

Physiological states of an organism and alterations of those states are often reflected in biological signals at a molecular level. For example, when certain hormones bind to corresponding protein receptors, the proteomic profile of an organism's cell and the function of the organism's bioelectric signals, such as the action potentials and function of the motoneurons transducing electrical signals to mechanical signals, can be altered. Other examples of biological conditions that similarly effect bioelectric signals include blood glucose levels, retinopathy, neurological, blood alcohol content, and ophthalmological disorders, among others.

Bioelectric potentials observed within the cells of the eye and/or on or near skin near the eye, for example, can include electrooculograms (EOGs), electroretinograms (ERGs), electroencephalograms (EEGs), electrocardiogram (EKGs), electromyograms (EMG), and others. These bioelectric potentials can be meaningful in view of baseline data for an individual organism or in view of standardized baselines established by research. The example techniques can, for example, correlate an EOG signal to blood glucose levels and/or blood alcohol content.

The retina is one of the most metabolically active sites in the body. The retina is separated from the blood stream by the blood retinal barrier which consists of three layers: retinal pigment epithelium (RPE), the Bruchs brane, and the choriocapillaris endothelium. The retina relies on glucose for its metabolic needs. The epithelia of the blood retinal barrier allow very little glucose to traverse the paracellular spaces composing the epithelia, unlike systemic endothelia. Glucose transport from the blood stream to the retina is therefore performed via transcellular mechanisms. As the retina is so metabolically active it requires both passive and active transporters. The passive transporters use diffusive forces to drive transport glucose, which can cause the flow of transporters [YK1] to be limited due to transporter saturation and diffusive transport requirements.

The active transporters only start to function when the passive transporters cannot keep up with the levels of glucose either needed by the retina or in the blood. These active transporters use charged ionic species, like sodium, to facilitate pumping the glucose across the membrane. This ion-coupled transport associates glucose transport with specific ion flow.

Moreover, acts as a crucial stimulus in the retina. When light is incident on the retina the photoreceptors in the eye register reception of the light and initiate a signal cascade. As a part of this cascade a substance referred to as the “light rise” or “light peak” substance traverses the intercellular space between the photoreceptor cells to the RPE cells which lay below them. The “light peak” substance initiates a signal cascade in the RPE cells upon arrival, which leads to the excretion of chloride ions on the basal side. This response to light is termed the light rise or light peak. Moreover a similar phenomenon can be observed by the removal of light from the system and the bioelectric response can be similar but opposite in sign. The magnitude of the signal can be observed to be logarithmically proportional to the retinal illuminance.

The presence or lack of sodium ions around the chloride ion transporters affect the amount of chloride ions secreted. If sodium ions are tied up in glucose transport they will exert less of an effect on the chloride transporters. This relationship suggests a correlative relationship where more glucose ties up more sodium which effects chloride transporters less allowing them to secrete more chloride. In summary, the amount of chloride ions the cells expel is altered as the levels of glucose in the blood stream change. This expulsion of chloride ions also creates a bioelectric potential originating in the eye. The RPE is located in on the retina and is itself the source of the charge difference that creates what is termed the “Cornea-Fundal potential” which can be measured as an electrooculogram.

In some examples, EOG signals are altered if a person has a lazy eye. If an eye is lazy it will require a different amount of electrical stimulation than that of a normal eye. If the parameters for the EOG shift due to an eye being lazy is established one simply has to observe a filtered EOG to see if and by how much a person's eye is lazy. In various examples, the techniques can detect different neurological disorders from various sections and frequencies of the EOG. Similarly, the techniques can use ERGs, EEGs, EKGs, and EMGs to detect similar or other biological conditions.

The techniques described herein can be implemented in a number of ways. Example implementations are provided below with reference to the following figures. The implementations, examples, and illustrations described herein can be combined.

The term “techniques” can refer to system(s), method(s), computer-readable media encoded with instructions, module(s), and/or algorithms, as well as hardware logic (e.g., Field-programmable Gate Arrays (FPGAs), Application-Specific Integrated Circuits (ASICs), Application-Specific Standard Products (ASSPs), System-on-a-chip systems (SOCs), Complex Programmable Logic Devices (CPLDs)), etc. as permitted by the context described above and throughout the document.

### Example Environment

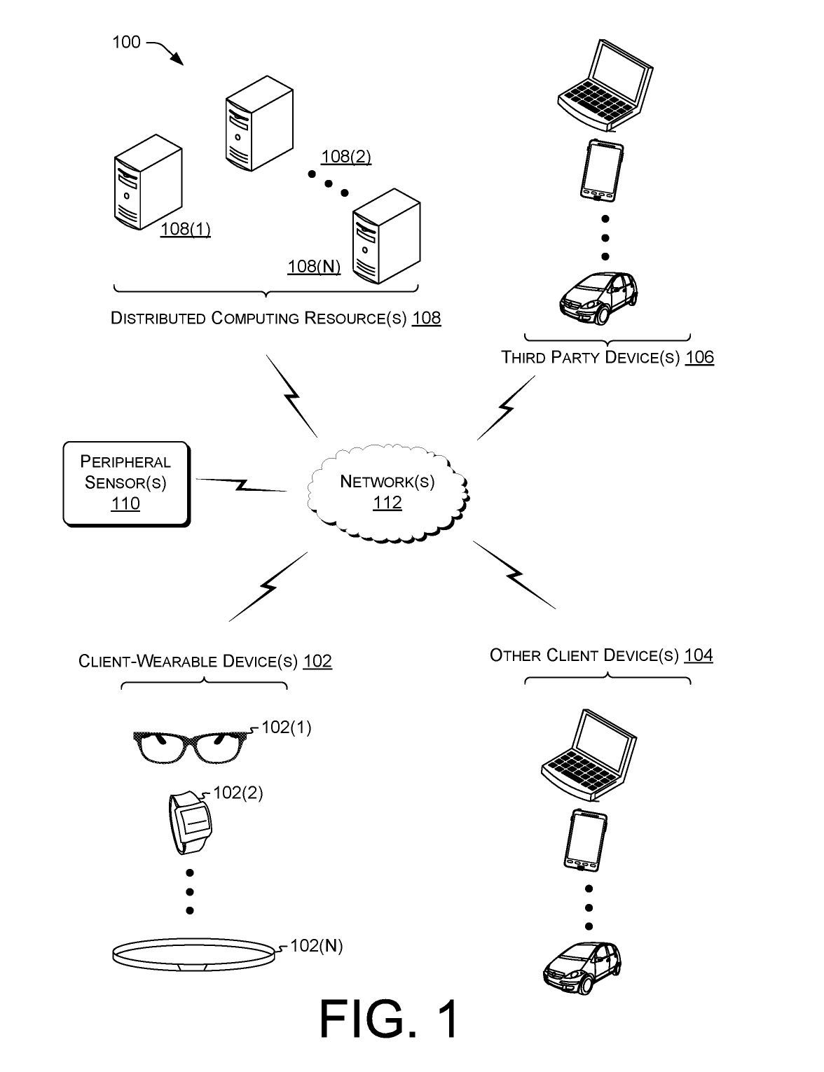

FIG. 1 is a block diagram depicting example environment 100 in which examples described herein can operate. In some examples, the various devices and/or components of environment 100 include client-wearable device(s) 102 that can communicate with one another and with one or more of other client device(s) 104, third-party device(s) 106, distributed computing resource(s) 108, and peripheral sensor(s) 110 via one or more networks 112 or other communicative coupling 106.

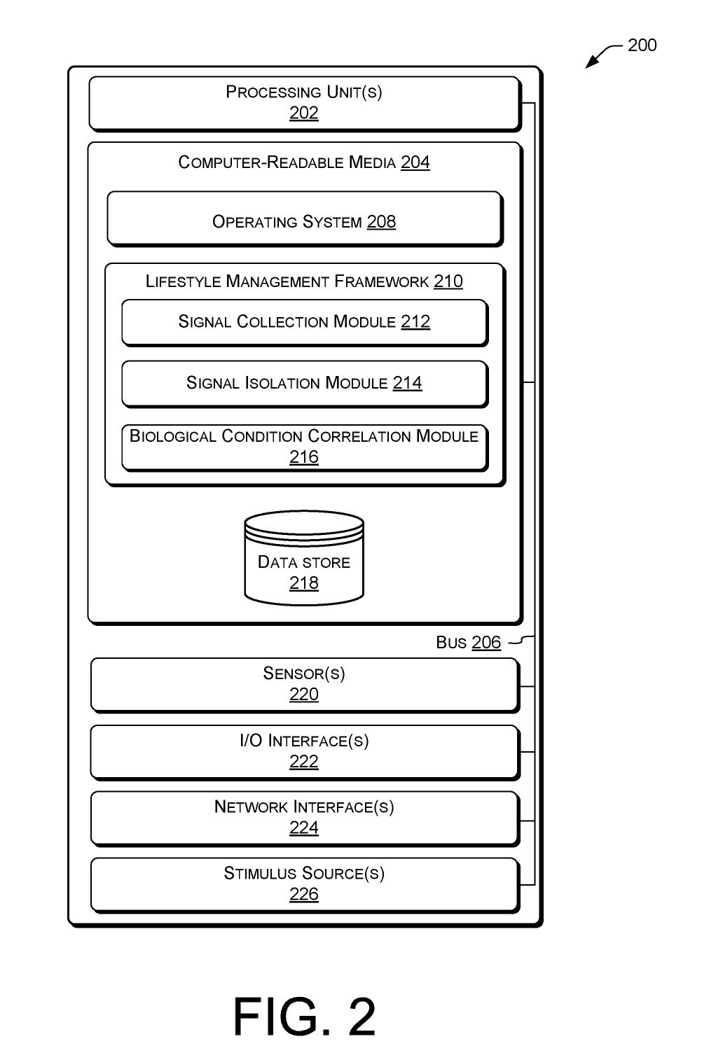

In at least one example, client-wearable device(s) 102, such as devices 102(1)-102(N) can include any item that a user can wear or that can persistently be close enough t e user to provide regular monitoring. In various examples, the client-wearable device(s) 102 include eyeglasses 102(1) and/or other head-mounted objects, a watch 102(2) and/or wrist band, a chest strap 102(N), a headset, Microsoft HoloLens, Google Glass, a hat, a belt, a glove, a sock, a shoe component, etc. As discussed in more detail in FIG. 2, the client-wearable device(s) 102 include one or more processors, network interface(s), computer-readable media, and/or sensor(s) to provide non-invasive bioelectric lifestyle management such as, for example, biological conditions, biological baseline data, and/or bioelectric signals, among others. In at least one example, the client-wearable device(s) 102 can store lifestyle management information at the client-wearable device(s) 102. In some examples, the client-wearable device(s) 102 can communicate lifestyle management information to one or more of the other client device(s) 104, third-party device(s) 106, and/or distributed computing resource(s) 108. In some examples, the client-wearable device(s) 102 can receive bioelectric, biological, and/or other signals from the peripheral sensor(s) 110 via network(s) 112.

In some examples, the other client device(s) 104 and the third party device(s) 106 can include, but is not limited to, desktop computers, server computers, web-server computers, personal computers, mobile computers, laptop computers, tablet computers, wearable computers, implanted computing devices, telecommunication devices, automotive computers, network enabled televisions, thin clients, terminals, personal data assistants (PDAs), game consoles, gaming devices, work stations, media players, personal video recorders (PVRs), set-top boxes, cameras, integrated components for inclusion in a computing device, appliances, and/or any other sort of computing device such as one or more separate processor device(s), such as CPU-type processors (e.g., micro-processors). GPUs, and/or accelerator device(s). In at least one example, the client-wearable device(s) 102 and the other client device(s) 104 can be associated with at least one user for which the techniques provide lifestyle management. In at least one example, the third party device(s) 106 can provide access to the lifestyle management information for the at least one user for medical professionals, relatives, and/or other parties given access to the lifestyle management information.

In various examples, distributed computing resource(s) 108 include computing devices such as devices 108(1)-108(N). Examples support scenarios where device(s) 108 can include one or more computing devices that operate in a cluster and/or other grouped configuration to share resources, balance load, increase performance, provide fail-over support and/or redundancy, and/or for other purposes. Although illustrated as desktop computers, distributed computing resource(s) 108 can include a diverse variety of device types and are not limited to any particular type of device. For example, distributed computing resource(s) 108 can include any type of computing device having one or more processing unit(s) operably connected to computer-readable media, I/O interfaces(s), and network interface(s). In at least one example, the distributed computing resource(s) 108 can store lifestyle management data and facilitate access thereto by the client-wearable device(s) 102, the other client device(s) 104, the third party device(s) 106, and the peripheral sensor(s) 110.

The system can further include peripheral sensor(s) 110 communicatively coupled to the network(s) 112. In various examples, peripheral sensor(s) 110 can include device(s) that capture, store, and/or transmit biological signals, bioelectric signals, and/or other signals. For example, the sensor(s) 110 can include a camera e.g., an infrared and/or visible light camera), electrode(s), electrocardiogram, endoscope, tonometer, retinoscope, toric marker, phoropter, blood pressure monitor, dialysis pressure sensor, breathing sensor, sound pressure sensor, pedometer, GPS, heart rate monitor, etc. In at least one example, the techniques use data from the peripheral sensor(s) 110 to isolate a particular bioelectric signal corresponding to a biological condition from a collected bioelectric signal, wherein the biological condition is derived from a known biological function. The type of peripheral sensor(s) 110 can be chosen to assist in accurate isolation of the desired bioelectric signal. For example, in at least one example, when a user desires to non-invasively ascertain blood glucose levels (i.e., one type of biological condition) of the user via EOG analysis, in order to isolate the EOG from other bioelectric signals present in a bioelectric signal collected by the client-wearable device(s) 102, a camera (i.e., one type of peripheral sensor 110) can provide data regarding gaze direction. In at least one example, the techniques can isolate the EOG from the other bioelectric signals in part based on the gaze direction at a certain time.

In some examples, network(s) 112 can include public networks such as the Internet, private networks such as an institutional and/or personal intranet, or some combination of private and public networks. Network(s) 104 can also include any type of wired and/or wireless network, including but not limited to local area networks (LANs), wide area networks (WANs), satellite networks, cable networks, Bluetooth, near field communication (NFC), Wi-Fi networks, WiMax networks, mobile communications networks (e.g., 3G, 4G, and an forth) or any combination thereof. Network(s) 112 can utilize communications protocols, including packet-based and/or datagram-based protocols such as internet protocol (IP), transmission control protocol (TCP), user datagram protocol (UDP), and/or other types of protocols. Moreover, network(s) 112 can also include a number of devices that facilitate network communications and/or form a hardware basis for the networks, such as switches, routers, gateways, access points, firewalls, base stations, repeaters, backbone devices, and the like.

In some examples, network(s) 112 can further include devices that enable connection to a wireless network, such as a wireless access point (WAP). Examples support connectivity through WAPs that send and receive data over various electromagnetic frequencies (e.g., radio frequencies), including WAPs that support Institute of Electrical and Electronics Engineers (IEEE) 1302.11 standards (e.g., 1302.11g, 1302.11n, and so forth), and other standards. In various examples the networks(s) 112 can include computer busses such as USB, IEEE 1394, or Lightning.

### Example Device

FIG. 2 depicts an example device 200, which can represent client-wearable device(s) 102. Example device 200 can include any type of computing device having one or more processing unit(s) 202, operably connected to computer-readable media 204, such as by bus 206. Processing unit(s) 202 can represent, for example, a CPU incorporated in device 200. The processing unit(s) 202 can similarly be operably connected to computer-readable media 204. In some examples, bus 206 can include one or more of a system bus, a data bus, an address bus, a PCI bus, a Mini-PCI bus, and any variety of local, peripheral, and/or independent buses, or via another operable connection, such as network 112.

The computer-readable media 204 can include, at least, two types of computer-readable media, namely computer storage media and communication media. Computer storage media can include volatile and non-volatile, non-transitory machine-readable, removable, and non-removable media implemented in any method or technology for storage of information (in compressed or uncompressed form), such as computer (or other electronic device) readable and/or executable instructions, data structures, program mod lies, and/or other data to perform processes or methods described herein. The computer-readable media 112 and the computer-readable media 122 can be examples of computer storage media. Computer storage media includes, but is not limited to hard drives, floppy diskettes, optical disks, CD-ROMs, DVDs, read-only memories (ROMs), random access memories (RAMs), EPROMs, EEPROMs, flash memory, magnetic and/or optical curds, solid-state memory devices, and/or other types of physical machine-readable media suitable for storing electronic instructions.

In contrast, communication media can embody computer-readable instructions, data structures, program modules, and/or other data in a modulated data signal, such as a carrier wave, and/or other transmission mechanism. As defined herein, computer storage media does not include communication media.

Device 200 can include, but is not limited to, desktop computers, server computers, web-server computers, personal computers, mobile computers, laptop computers, tablet computers, wearable computers, implanted computing devices, telecommunication devices, automotive computers, network enabled televisions, thin clients, terminals, personal data assistants (PDAs), game consoles, gaming devices, work stations, media players, personal video recorders (PVRs), set-top boxes, cameras, integrated components for inclusion in a computing device, appliances, and/or any other sort of computing device such as one or more separate processor device(s), such as CPU-type processors (e.g., micro-processors), GPUs, and/or accelerator device(s). In at least one example, the device 200 includes eyeglasses and/or other head-mounted objects, a watch and/or wrist band, a chest strap, a headset, Microsoft HoloLens®, Google Glass®, a hat, a headband including conductive fabrics a belt, a glove, a sock, a shoe component, etc.

In some examples, as shown regarding device 200, computer-readable media 204 can store instructions executable by the processing mitts) 202, which can represent a CPU incorporated in device 200. Computer-readable media 204 can also store instructions executable by an external CPU-type processor, executable by a GPU, and/or executable by an accelerator, such as a Field Programmable Gate Array (FPGA)-type accelerator, a digital signal processing (DSP)-type accelerator, and/or any internal or external accelerator.

Executable instructions stored on computer-readable media 202 can include, for example, an operating system 208, a lifestyle management framework 210, and other modules, programs, and/or applications that can be loadable and executable by processing s(s) 202. The lifestyle management framework 210 can include signal collection module 212, signal isolation module 14, and biological condition correlation module 216.

Although FIG. 2 depicts modules 212-216 as being modules stored on computer-readable media 202, part or all of the modules can be implemented in hardware. For example, the signal collection module 212 can be a nix of hardware to receive a signal and instructions to transmit or store the received signal. In this example, the signal collection module 212 can include an analog/digital converter to convert the received sign to an analog or digital signal respectively. In some examples, signal isolation module 214 can include filtering and/or amplification components implemented in hardware or instructions stored on the computer-readable media 204. In this example, the signal isolation module 214 can be implemented in hardware if the received signal is an analog signal, instructions on the computer-readable if the received signal is a digital signal or is converted to a digital signal, and/or some combination thereof.

Moreover, the biological condition correlation module 216 can be implemented in hardware. For example, in an example where an isolated bioelectric signal needs to be converted to a meaningful indication of a biological condition (e.g., a glucose level measurement, a heart rate, indication of brain activity, blood alcohol content etc.) the biological condition correlation module 216 can be implemented in either hardware or software to accomplish the conversion. As a clarification, a biological condition can be meaningful as data presented in recognizable standard units and/or in a humanly accessible way such as in a graph or other visualization. For example, although a voltage of a bioelectric potential may correspond to a particular glucose level, that voltage may be meaningless to a user or medical professional. In that example, the biological condition correlation module 216 can, by hardware, software, or a combination thereof, correlate and/or convert the bioelectric signal to a meaningful metric of blood glucose levels, such as mmol/L or mg/dL.

In some examples, the functionality described herein can be performed, at least in part, by one or more hardware logic components such as accelerators. For example, and without limitation, illustrative types of hardware logic components that can be used include FPGAs, Application-specific Integrated Circuits (ASICs), Application-specific Standard Products (ASSPs), System-on-a-chip systems (SOCs), Complex Programmable Logic Devices (CPLDs), etc. For example, an accelerator can represent a hybrid device, such as one from XILINX or ALTERA that includes a CPU core embedded in an FPGA fabric.

Furthermore, any of these functions can be accomplished at another device, such as the other client device(s) 104, third-party device(s) 106, and/or distributed computing resource(s) 108. In order for these functions to be accomplished at another device, the device 200 can communicate the collected signal, isolated signal, biological condition or any prerequisite or intermediate data or signal to the other device.

In some examples, computer-readable media 204 also includes a data store 218. In some examples, data store 218 includes data storage such as a database, data warehouse, and/or other type of structured or unstructured data storage. In some examples, data store 218 includes a relational database with one or more tables, indices, stored procedures, and so forth to enable data access. Data store 218 can store data for the operations of processes, applications, components, and/or modules stored in computer-readable media 204 and/or executed by processor(s) 202, and/or accelerator(s), such as signal collection module 212, signal isolation module 214, or biological condition correlation module 216. For example, data store 218 can store version data, iteration data, clock data, and other state data stored and accessible by the lifestyle management framework 210. Alternately, some or all of the above-referenced data can be stored, processed, and/or further processed at the other client device(s) 104, third-party device(s) 106, and/or distributed computing resource(s) 108 or at an external CPU-type processor (e.g., microprocessor(s)), memory on board a GPU, memory on board an FPGA type accelerator, memory on board a DSP type accelerator, and/or memory on board another accelerator).

The device 200 can further include sensor(s) 220. In at least one example, the sensor(s) 220 include an electrode (e.g., a capacitance-driven electrode, an impedance-driven electrode, etc). The sensor(s) 220 can include any device capable of non-invasively sensing bioelectric potentials, whether via, an organism's skin, near the organism's body, through clothes on the organism's body, or any other method. In some examples, the sensor(s) 220 also include at least one of an ambient light sensor, a camera, or any other device that can provide contextual data to help facilitate isolation of a particular bioelectric signal of interest by the signal isolation module 214. In some examples, the sensor(s) 220 can be integrated into the device 200. In additional or alternative examples, one or more sensors of the sensor(s) 220 can be a peripheral sensor 110. In various examples, the sensor(s) 220 can be integrated into a conductive textile composing at least part of a headband. The sensor(s) 220 and lifestyle management framework 210 can be designed to sample a biological condition at a point in time or continuously. The lifestyle management framework 210 can also provide the biological condition data as a single data point, a collection of data points, or a continuous stream of data, whether by the I/O interface(s) 222 or via the network interface(s) 224.

In some examples, device 200 can further include one or more input/output (I/O) interface(s) 222 to allow device 200 to communicate with input/output devices such as user input devices including peripheral input devices (e.g., a keyboard, a mouse, a pen, a game controller, a voice input device, a touch input device, a gestural input device, Kinect, and the like) and/or output devices including peripheral output devices (e.g., a display, a printer, audio speakers, a haptic output, bone conduction for audio sensation, and the like). In at least one example, the I/O interface(s) 222 can be used to communicate with sensor(s) 220, whether the sensor(s) 220 are integrated into the device 200 or are peripheral. In some examples, device 200 can also include one or more network interface(s) 224 to enable communication between device 200 and other networked devices such as the other client device(s) 104, third-party device(s) 106, and/or distributed computing source(s) 108. Such network interface(s) 224 can include one or more network interface controllers (NICs) and/or other types of transceiver devices to send and receive communications over a network, such as network(s) 112. In at least one example, the I/O interface(s) 222 can interface with at least one of a microphone or a speaker. In some examples, the device 200 can relay instructions to conduct a biological condition test and/or biological condition results. For example, in an example where an EOG or ERG is the bioelectric signal of interest, the device 200 can instruct a user via the speaker to look in a particular direction and/or can re-instruct a user if an error occurs.

In at least one example, device 200 can also include stimulus source(s) 226. Depending on the biological condition desired to be evaluated, a stimulus may cause or augment a bioelectric response of the organism or facilitate isolation of the bioelectric potential. The stimulus source(s) 226 can include light(s), a device to cause pain, and/or electrode(s) to supply a voltage at an area of the organism, for example.

For example, to measure blood glucose levels from an EOG, the stimulus source(s) can include a light source, such as a light-emitting diode (LED), that the signal collection module 212 activates to stimulate the retinal cells of an eye, causing them to dump chloride ions in proportion with a blood glucose level, thereby hyperpolarizing and causing a bioelectric potential in the eye and detectable by the techniques. In at least one example, the LED can emit blue and/or green light. In some examples, the LED can emit light of a different frequency. In yet other examples, the stimulus source(s) 226 can emit light outside the visible light spectrum alternatively or additionally.

As discussed above, the increased metabolic requirements of the eye cause retinal cells to expel chloride ions in proportion to blood glucose levels. In some examples, since eyes sense light logarithmically and because hyperpolarization of retinal cells occurs as a response to a difference in light contacting the cells, the stimulus source(s) 226 can include a light source and the sensor(s) 220 can include an ambient light sensor. The signal collection module 212 can use these components to activate the light source in proportion to ambient light of an environment of the device 200. In other words, the signal collection module 212 can be configured to increase and decrease the luminosity of the light source depending on ambient light intensity to provide a sufficient difference between the light source luminosity and ambient light intensity in order to facilitate more easily isolating the EOG. In at least one example, the light source(s) 226 can be disposed on a nose bridge of eyeglasses. In some examples, the light source(s) 226 can be additionally or alternatively disposed elsewhere on or in the device 200.

FIG. 2 includes an example lifestyle management framework 210 that can be distributively or singularly stored on device 200, which, as discussed above, can include one or more devices such as client-wearable device(s) 102, the other client device(s) 104, the third party device(s) 106, or the distributed computing resource(s) 108. Some or all of the modules can be available to, accessible from, or stored on a remote device, such as a cloud services system or distributed computing resource(s) 108, and/or device(s) 102-106. In at least one example, an image interpretation framework 210 includes modules 212-216 as described herein that provide non-invasive bioelectric lifestyle management, by the signal collection module 212, the signal isolation module 214, and the biological condition correlation module 216. In some examples, any number of modules could be employed and techniques described herein as employed by one module can be employed by a greater or lesser number of modules.

In at least one example, the signal collection module 212 controls current provided to electrode(s) composing the sensor(s) 220 to be able to sense bioelectric signals. The signal collection module 212 can be configured to include a signal processing module that removes one or more of noise, motion artifacts, saccade movements, and unwanted bioelectric signals (e.g., EMGs and EKGs if the techniques are isolating EOGs, etc.) from the collected bioelectric signals. The signal processing module can further include anti-aliasing.

In at least one example the signal processing module is an analog signal processing module. In various examples, the signal processing module is a mixed analog and digital signal processing module including an analog-to-digital converter or, in other examples, it is a digital signal processing module. The signal collection module 212 can also further include a protective circuit that prevents current from going from the electrodes to the rest of the device 200 and thus to the person.

In at least one example, the lifestyle management framework 210 also includes a signal isolation module 214 which is configured to isolate a particular bioelectric signal from the signal collected by the signal collection module 212. Since different types of bioelectric signals are present in different frequency and power spectra, depending on the biological condition of interest and its corresponding bioelectric signal, the signal processing module can further include filters and amplifiers that respectively filter and amplify a particular frequency spectrum which is known to contain the bioelectric signal of interest. For example, in an application where the biological condition of interest is blood glucose levels and the EOG is therefore the bioelectric signal of interest, EOGs tend to have an amplitude of 5-10 μV and the signal collection module 112 can include a band-pass or similar filter that passes a 0.5-30 Hz spectrum. In some examples, the filter can pass a 0.5-35 Hz spectrum for an EOG.

For an example where an EKG is the bioelectric signal of interest, the dominant range is 2.5-10 Hz but the whole signal may be in the range of 0.67-40 Hz therefore the filter can pass a 2.5-10 Hz spectrum or a 0.5-40 Hz spectrum. For ERGs, the filter can pass a 25-35 Hz spectrum. For an EEG, the filter can pass a 0.5-70 Hz spectrum where the alpha wave is in the spectrum of 8-15 Hz, the beta wave is in the spectrum of 16-31 Hz, the gamma wave is in the spectrum of 32-70 Hz, the delta wave is in the spectrum of 0.5-4 Hz, the theta wave is in the spectrum of 4-7 Hz, and the mu wave is in the spectrum of 7-20 Hz. In some examples, where the EEG is the bioelectric signal of interest, the signal collection module 212 can pass the alpha wave, beta wave, gamma wave, delta wave, theta wave, and mu wave spectrums separately or in separate channels. For EMGs, the filter can pass a 7-20 Hz spectrum.

In some examples, the signal collection module 212 or the signal isolation module 214 can include further amplifiers, such as instrumentation amplifiers, for the purposes of removing noise (e.g., noise from one or more of movement of sensors relative to skin, bioelectric signals other than the desired bioelectric signals) and artifacts and amplifying bioelectric signals that are known to be low power although other amplifiers that remove noise and artifacts while signal gain can be increased. In at least one example where the biological signal of interest is an EOG or ERG, a common-mode rejection ratio of an amplifier amplifying the signal can be 100 dB and the gain can be 100,000. In some examples, the gain can be between 50,000 and 100,000. In various examples, when an EOG is the signal of interest, an EOG can have a magnitude of approximately 1-10 microvolts. In various examples, the gain can be chosen to amplify that signal so that is has a magnitude in the millivolts. In other examples, other gains can be chosen. In at least one example, the signal isolation module 214 can use a two-step amplification process in which common mode noise is attenuated, followed by a large amplification of the signal to scale the signals to a range that facilitates visualization and analysis.

Moreover, the signal isolation module 214 can use data received from other sensor(s) 220, stimulus source(s) 226, and/or peripheral sensor(s) 110, etc., to increase accuracy of isolation of the bioelectric signal of interest. For example, when analysis of an EOG or ERG is desired, data from a camera (i.e., another sensor(s) 220) can be used to track the gaze of one or more eyes. This gaze data can be correlated with fluctuations in the collected signal corresponding to movement of the eye, both to identify the relative level EOG or ERG and to remove noise due to the movement itself. In at least one example, the device 200 can provide instructions via the I/O interface(s) 224 via a display or a speaker, for example, or another device with which the device 200 is in communication via network interface(s) 224 can provide instructions to a user directing them to look in a certain direction. In some examples, these instructions could be given coincident with gaze tracking and stimulus, such as light, or upon confirmation by gaze tracking that the user has looked toward the light source. In some examples, the signal isolation module 214 can use information related to stimulus provided to the organism by the stimulus source(s) 226, such as a temporal duration relative to the collected signal and/or a magnitude of the stimulus provided. In various examples, the signal isolation module 214 can use data specifying an ambient light intensity of the environment of the user from an ambient light sensor and a camera (i.e., composing sensors 220) to isolate an EOG or ERG from the collected signal. In various examples, the data received from other sensor(s) 220, stimulus source(s) 226, and/or peripheral sensor(s) 110, etc. can comprise temperature information from an infrared camera or other thermal sensor, audio information from a microphone, or heart rate, among others.

In at least one example, the lifestyle management framework 210 can also include a biological condition correlation module 216 which is configured to convert biological signals to a meaningful indication of a biological condition. For example, in an example where an isolated bioelectric signal needs to be converted to a meaningful biological condition (e.g., a glucose level measurement, a heart rate, indication of brain activity etc.) the biological condition correlation module 216 can be implemented in either hardware or software to accomplish the conversion. As discussed above, an indication of a biological condition is meaningful when it is presented as data of recognizable standard units and/or in a graph or other visualization that aids a user to understand the information relayed. In some examples, the format of the data produced by the biological condition correlation module 216 can be different depending on who the biological condition data is to be sent to. For example, a user of the device 200 can receive a more simplified indication of the biological condition e.g., “good,” “ask your doctor if these results merit further testing,” “seek medical assistance immediately”) whereas a medical professional or developer can receive more detailed information regarding one or more of the biological condition, the underlying bioelectric signal, or the collected signal. In some examples, the device 200 can contact emergency medical assistance, with or without the user's authorization depending on the determined rest its of the biological condition correlation.

In at least one example, a the biological condition correlation module 216 can measure a nystagmus of an eye using data from one or more sensors or peripheral sensors and correlating the nystagmus to one or more biological conditions such as, for example, toxicity, congenital disorders, nervous system disorders, pharmaceutical drug effects, blood alcohol content, or rotational movement.

### Example Environment

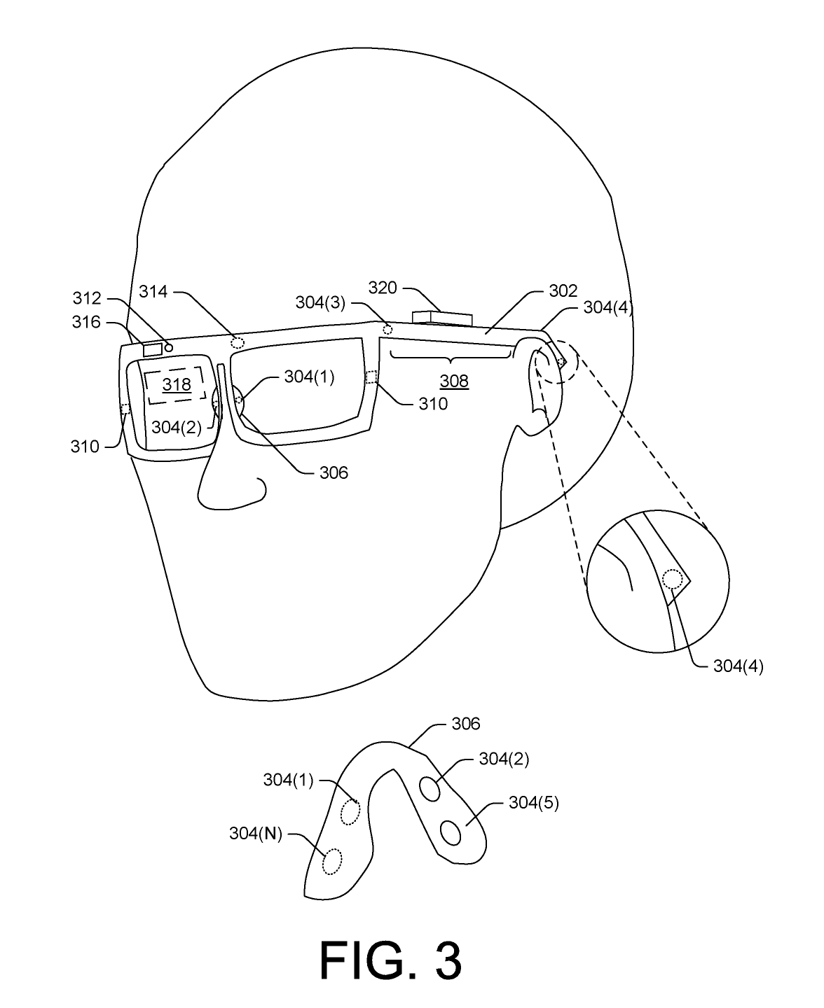

FIG. 3 is an example environment 300 in which the techniques can be employed. FIG. 3 is not to scale. The example environment 300 can include a device 302, such as a device 200 that implements non-invasive bioelectric lifestyle management. In at least one example, the device 302 can include eyeglasses or a similar head-mounted device such as, for example, a head strap, a hat, Microsoft HoloLens®, or Google Glass®, among others. In some examples, the device 302 can include lenses, but in other examples the device 302 is an eyeglass frame or similar structure.

In some examples, the device 302 can include one or more electrode(s) 304(1)-304(N), which can act as the sensor(s) 220 of FIG. 2. FIG. 3 illustrates a plurality of possible locations at which the one or more electrode(s) 304(1)-304(N) can be located to collect bioelectric signals from a human user. For example, one or more of the electrode(s) 304(1)-304(N) can be disposed on the nasal side of a nose bridge 306 of the eyeglasses in a position configured to contact or be near the skin at or near the medial canthus of the eye, such as the positions of electrodes 304(1) and 304(2). In some examples, the bioelectric signal of interest can be collected at a location different from the location at which the bioelectric signal was generated since electric signals propagate through the body. Additionally or alternatively, one or more of the electrode(s) 304(1)-304(N) can be disposed along a lateral (or, equivalently, temple) arm 308 of the eyeglasses, such as electrode 304(3), so as to contact or be near the skin on or near the lateral canthus of the eye of the user or a temple of the user. Although only 304(3) is shown in FIG. 3, in some examples, a second or more electrodes (not illustrated) can be in a similar position on the opposite lateral arm of the eyeglasses.

In at least one example, the device 302 can include one or more electrodes to monitor a first eye, one or more electrodes to monitor a second eye, and one or more electrodes to act as ground. In this example, the bioelectric signal can be isolated based at least in pail on a signal that is common to at least some of the electrodes. In this and other examples, the signals collected by the electrodes can be compared and/or averaged.

In at least one example, the device 302 can include one or more electrodes 304(4) to function as grounding electrode(s). In some examples, the grounding electrode(s) 304(4) can be positioned on the temporal bone behind the ear. This position helps filter out EEGs and other physiologic noise, which can he desirable in some cases but undesirable in others. Electrode(s) 304(5) and 304(N) illustrate alternate or additional positions of electrode within the nose bridge 306 of the eyeglasses. In at least one example, electrodes 304(1), 304(2), 304(5), and 304(N) can all be used to collect bioelectric signals.

In some examples, the electrode(s) 304(1)-304(N) are impedance driven electrode(s), in which case, the electrode(s) 304(1)-304(N) contact the skin. In at least one example, the electrode(s) 304(1)-304(N) are capacitance driven and need only be near enough to the skin for the capacitance of the electrode(s) 304(1)-304(N) to be affected by the electric field of bioelectric potentials enough to collect the bioelectric signal of interest. Therefore, in some examples, electrodes 304(1)-304(N) may collect the bioelectric signal without direct contact with the skin and through the clothes, hair, and/or other intermediate material between the electrode and the skin.

In some examples, the device 302 can include stimulus source(s) 310, such as stimulus source 226. In at least one example the stimulus source(s) 310 can include one or more LEDs. Although FIG. 3 depicts stimulus source(s) 310 as being disposed on outward lateral sides of rims of the eyeglasses, the stimulus source(s) 310 can be disposed anywhere within or outside view of the user. If the stimulus source(s) 310 include alight source for causing or augmenting isolation of EOGs or ERGs, the stimulus source(s) 310 should be disposed where light from the light source will be able to contact the retina. In some examples, the stimulus source(s) 310 can be disposed the lower portion of the rim, upper portion of the rim, and/or along the lateral arm 308.

In some examples, the device 302 can further include an ambient light sensor and/or camera 312 which can fulfill the functionality of a sensor of the sensor(s) 220, as discussed above. In at least one example, the ambient light sensor and/or camera 312 can be used to adjust the magnitude of stimulus provided by the stimulus source(s) 310. In some examples, ambient light data received by the ambient sensor can be used to help isolate a particular bioelectric signal. In some examples, a camera 312 can be used to provide experimental control data, such as to aid in color blindness and vision testing.

In some examples, the device 302 can further include a gaze tracking device 314, such as a camera or infrared sensor. The gaze tracking device 314 can supply gaze data to facilitate isolation of a bioelectric signal of interest. In some examples, the gaze tracking device 314 can additionally be used to provide thermal information.

In some examples, the device 302 can further include a display device 316 to provide information via a display area 318 on a lens of the eyeglasses. In various examples, the device 302 can use the display area to provide indications of biological conditions. The device 302 can additionally or alternatively communicate biological condition information or bioelectric signal data to another device for display at the other device. For example, a device 302 including the described eyeglasses can communicate bioelectric signal data via Bluetooth to a user's smartphone for one or more of digital signal processing, analysis, or display. The user's smartphone can then display simplified biological condition data and/or bioelectric signal data to the user, store the biological condition data and/or bioelectric signal data locally, transmit the biological condition data and/or bioelectric signal data to distributed computing resource(s) 108 for further processing or storage, and/or to third-party device(s) associated with a medical professional.

FIG. 3 also illustrates a computing component 320 that can include one or more processors 20 computer-readable media 204, an operating system 208, lifestyle management framework 210, I/O interface(s) 222, and/or network interface(s) 224, among others. In some examples, the computing component 320 can be clipped onto eyeglasses, a hat, or other objects described herein and the bus 206 can be adhesively or otherwise affixed to the eyeglasses, hat, or other objects. In other examples, the computing component 320 can be integrated into and/or onto the device 302 and the bus 206 can be built into, adhered to, or otherwise affixed the eyeglasses frame. For example, computing component 320 can be integrated within an arm of the device 302 such that it is not externally visible. In other examples, the bus 206 can include wireless networks. In some examples, the computing component 320 can further include a speaker and can positioned to facilitate conveying messages to the user.

In at least one example, one or more of the components 304, 310, 312, 314, and 316 are integrated into the device 302. In some examples, one or more of the components 304, 310, 312, 314, and 316 can be affixed to eyeglasses, a hat, or other object described herein.

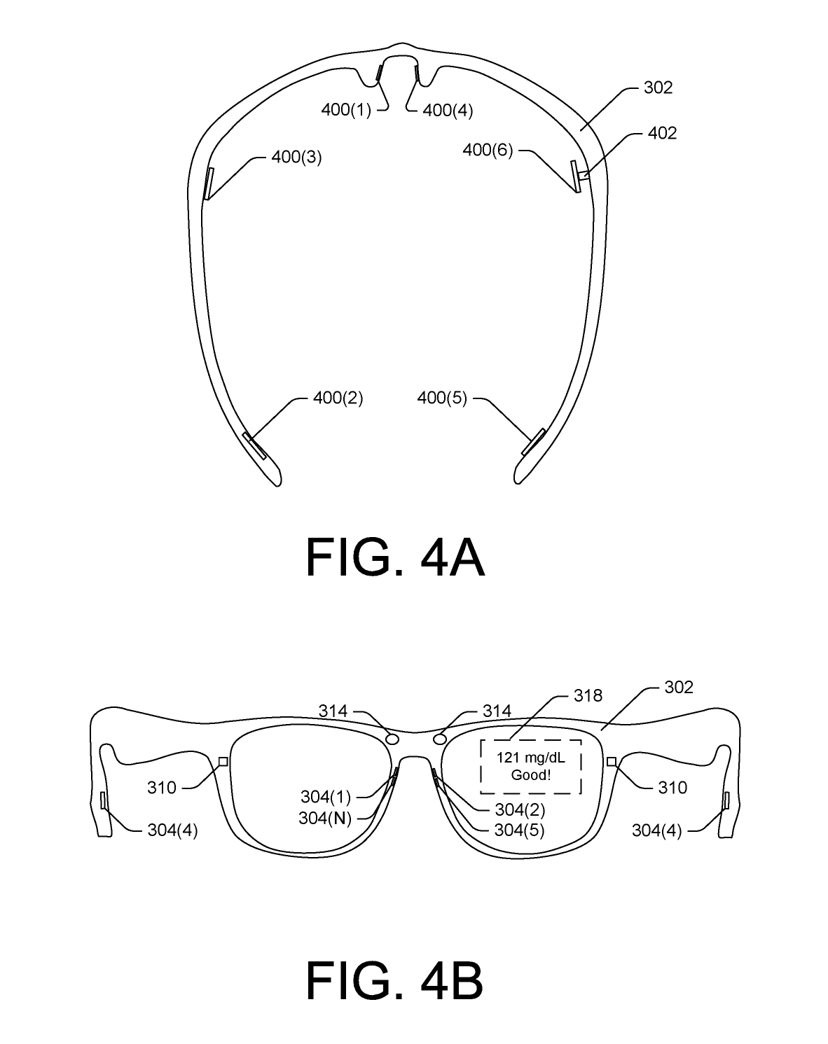

FIG. 44 illustrates a top view of the example environment 300 and various configurations of electrodes 400, such as electrodes 304(1)-(N) discussed above that can be included in device 302. FIGS. 4A and 4B are not to scale. In at least one example, one or more of the electrodes 304 can be arranged as electrodes 400(1) and 400(2), integrated in the device 302, such as in the frame of eyeglasses. The electrodes 400(1) and 400(2) can be inset in the frame and/or covered by part of the frame if the electrodes 400(1) and 400(2) are selected so that they are still capable of collecting the bioelectric signals of interest through the part of the frame. In other examples, one or more of the electrodes 304 can be mounted on an exterior of the device 302, such as electrodes 400(3), 400(4), and 400(5). In some examples, externally mounted electrodes may need to b disposed closer to or on skin of a user to detect a bioelectric signal of interest. In this example, further the device 302 can include an offset structure 402 to dispose an electrode 400(6) closer to or on skin of a user. In various examples, the electrode 400(6) can contact the lateral canthus. The particular locations of the electrodes 304 chosen can depend on the type bioelectric signal of interest, the type of electrode, aesthetics, and/or the power spectrum occupied by the bioelectric signal.

FIG. 4B illustrates an example view through the device 302 having a particular configuration of electrodes 304(1)-(N). In at least one example, the device 302 can have two cameras 314, as depicted. In at least one example, the device 302 can include two stimulus sources 310. In some examples, the device 302 can include four stimulus sources 310, where the two stimulus sources 310 not illustrated can be disposed near the cameras 314. FIG. 4B also illustrates an example output to display area 318. In the illustrated example, the display area 318 contains biological condition data (i.e., “121 mg/dL”) and simplified biological condition data (i.e., “Good!”). In some examples, the output to the display area 318 can include only the simplified biological condition data. In various examples, the display area 318 can pulse a color signifying a biological condition state such as, for example, green for a satisfactory state, yellow for a threshold state, and/or red for a state requiring attention. Although colors are discussed, any appropriate feedback to convey biological condition states is contemplated. In various examples, the stimulus source(s) 310 can provide this feedback. As used herein, a biological condition may be interchangeably referred to as a biological condition state.

### Illustrative Processes

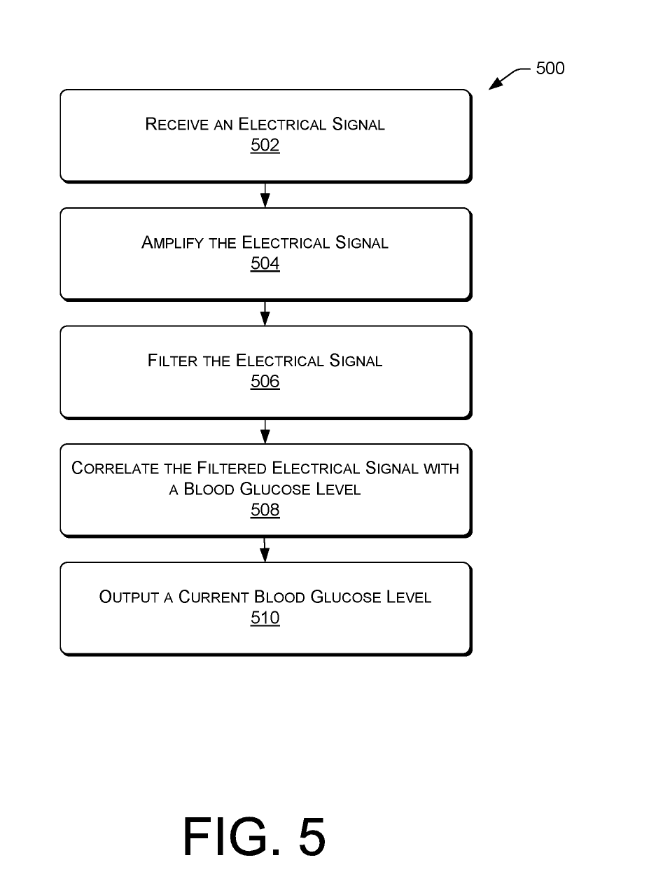

FIGS. 5-7 illustrate example processes 500, 600, and 700, which can be performed in whole or in part and singularly or in any combination.

FIG. 5 depicts an illustrative process 500 of implementing non-invasive bioelectric lifestyle management. At 502, the process receives an electrical signal, such as from sensor(s) 220 as discussed above. In at least one example, the process can, in conjunction with components of the client-wearable device(s) 102 and/or the other client device(s) 104, direct a user to look in a certain direction in order to increase accuracy of the received electrical signal. In some examples, the process also receives gaze tracking data in order to correlate the received electrical signal with a biological condition.

In some examples, the process can provide accurate results with gaze bucking and without needing to direct the user to look in a certain direction. In some examples, the process can illuminate a retina in conjunction with directions and/or when the process receives gaze tracking data that suggests that illumination of the retina may improve results. In various examples, the process can receive an electrical signal when gaze data indicates that a retina has maintained the same position for a threshold amount of time (e.g., 1 second, 3 seconds, 5 seconds, etc.) to process the electrical signal of interest. In some examples the gaze data can be used to measure a dilation of the iris. In various examples, the dilation of the iris can be used to identify biological conditions and/or an amplitude of stimulus to provide. Depending on the example implemented, the process can continuously, semi-continuously, or discretely measure a user's biological condition.

Moreover, since eyes are polarized from the retina to the cornea, the level of the potential, and therefore the EOG, can be affected by movements of the eye. In at least one example, the process can direct the user to look left and then to look right to cause a detectable bioelectric signal, in some examples, the process can detect the bioelectric signals from the eye based on the natural movements of the eye and without giving the user prompts.

At 504, the illustrative process 500 amplifies the received electrical signal. As discussed above, the device 200 or 302 can include analog signal processing to remove noise, aliasing, motion artifacts, saccade movements, and unwanted bioelectric signals and boost the resulting signal. For example, an anti-aliasing filter can reduce signals of higher frequency aliasing back to the frequency spectrum of the signals of interest. At 506, the illustrative process 500 filters the amplified electrical signals to remove electrical signals with a frequency above or below a predetermined threshold. In order to accomplish this, in at least one example a band-pass or similar filter can remove signals outside a frequency spectrum at which the signals of interest typically exist. In some examples, a low-pass and or high-pass filter can be used. Typical frequency spectra of various signals of interest are discussed above.

At 508, the illustrative process 500 correlates the filtered electrical signals to a blood glucose level of the user. In at least one example, the illustrative process 500 can correlate the filtered electrical signals with a blood glucose level by comparing the filtered electrical signal to a baseline value. In some examples, the baseline value is established by receiving the electrical signals, amplifying the electrical signals, and filtering the electrical signals and separately ascertaining a glucose level by another method, such as by using a lancet and blood glucose monitor or another blood glucose level measurement test. In at least one example, a user or the monitor itself provides the measured blood glucose level to the client-wearable device(s) 102 or the other client device(s) 104 and the measured blood glucose level is correlated with an amplitude of the filtered electrical signal. Variations of the amplitude of the filtered electrical signal therefrom can be correlated to a variation in blood glucose level. The client-wearable device(s) 102 and/or the other client device(s) 104 can notify the user to prompt recalibration. In some examples, the measured blood glucose level is stored in the computer-readable media of the other client device(s) 104 to serve as the baseline value. In various examples, the measured blood glucose level is stored in the computer-readable media of the client-wearable device(s) 102, the distributed computing resource(s) 108, and/or the third party device(s) 106.

In at least one example, the received electrical signal includes an EOG comprising an approximately square wave which corresponds to a saccade movement of at least one of the user's eyes. In some examples, the process fits a function to the filtered electrical signal in order to calculate an amplitude of the electrical signal. In some examples, the function comprises one or more of a square wave, a triangle wave, a sinusoid, and a constant. In at least one example, the techniques include a linear minimization of one or more of a square wave, a triangle wave, a sinusoid, and a constant to fit the wave. In various examples, any suitable line-fitting function can be used. In at least one example, an amplitude and frequency of the fitted function (e.g., biopotential data) can be used to determine blood glucose levels and/or blood alcohol content. In at least one example, the determination can be based at least in part on one or more of the change in light flux, a discrete time for which incident light was shone, calibration data for the organism, and baseline data from testing. In at least one example this amplitude is corresponded with a blood glucose level based at least in part on the baseline value.

For example, the process can calculate a difference in amplitude in the electrical signal from the amplitude of the electrical signal at the measured blood glucose level. The process can then use that difference to calculate a difference in blood glucose level from the measured (or baseline) glucose level. In some examples, the amplitude can he correlated to a blood glucose level without the baseline value. In this example, a correlation between biopotentials and glucose levels can be used. This correlation can occur at one or more of the client-wearable device(s) 102, the other client device(s) 104, the third party device(s) 106, and/or the distributed computing resource(s) 108.

At 510, the process outputs the current blood glucose level. In at least one example, the process can output the current blood glucose level visually and/or audibly at one or more of the client-wearable device(s), the other client device(s) 104, or the third party device(s) 106.

In some examples, the illustrative process 500 can include a step between 506 and 508 including converting the electrical signals from analog to digital. In at least one example, an A/D converter can accomplish this, whether the A/D converter and conversion exists at the device 200 or 302 or other client device(s) 104, third party device (s) 106, and/or distributed computing resource(s) 108. In at least one example the A/D converter exists at the device 200 or 302. In some examples, the A/D converter is part of other client device(s) 104. In other examples, a device can output the filtered (analog or digital) electrical signal without correlating the filtered electrical signal with a blood glucose level.

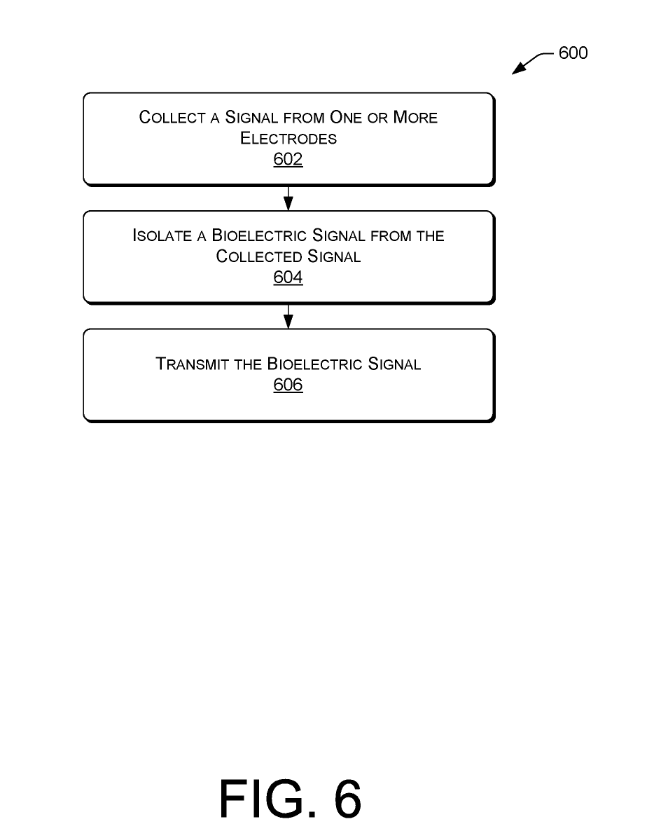

FIG. 6 depicts an illustrative process 600 of implementing non-invasive bioelectric lifestyle management. At 602, the process can collect at least one signal from one or more electrodes in any manner described herein. At 604, the process can isolate a bioelectric signal from the collected signal in any manner described herein. In at least one example, the process can amplify and filter the collected signal at a frequency and power spectrum corresponding to the bioelectric signal of interest, as discussed above. At 606, the process can transmit the bioelectric signal. In at least one example, the process can transmit the bioelectric signal from client-wearable device(s) 102 to one or more of other client device(s) 104, third-party device(s) 106, or distributed computing resource(s) 108. In some examples, the process can transform the bioelectric signal to biological condition data before transmitting. In other examples, the transform illusion can occur after transmission. In some examples, the process can transmit the bioelectric signal (and/or the biological condition data.) to a display and/or speaker of any of the devices discussed above.

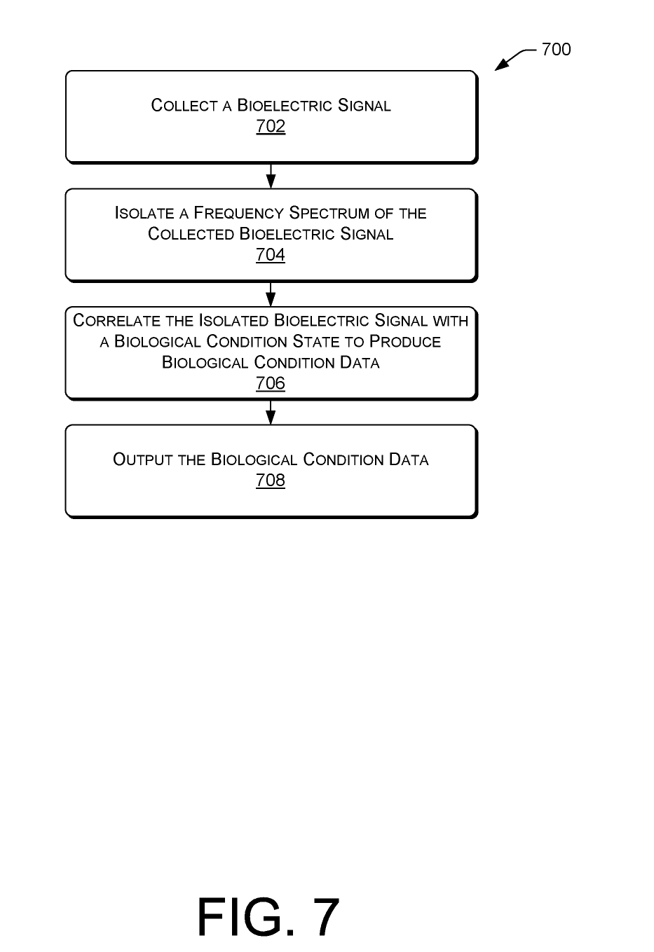

FIG. 7 depicts an illustrative process 700 of implementing non-invasive bioelectric lifestyle management. At 702, the process collects a bioelectric signal in any manner described herein. For example, the process can collect a bioelectric signal using electrodes disposed on or near one or more of skin near an eye, skin of a nasal bridge, skin of a temple, skin around or behind an ear, a lateral canthus of an eye, a medial canthus of an eye, or on a surface of an eye. At 704, the process can isolate a frequency spectrum of the collected bioelectric signal in any manner described herein.

At 706, the process can correlate the isolated bioelectric signal to a biological condition state in any manner described herein. For example, a baseline can be established through calibration of collected bioelectric signals with a known (e.g., measured using a separate device) bioelectric condition state. In that example, observed fluctuations in the bioelectric signals can be correlated with fluctuations in the bioelectric condition state. In at least one example, a biological condition state can be a particular condition at a specific time or range of time of the biological condition.

At 708, the process outputs the biological condition data in any manner described herein. For example, the process can transmit the biological condition data via a network; output the biological condition data to a display as a number, word(s), graph, visualization, or any combination thereof; output the biological condition data to a speaker for reproduction; etc.

### Example Clauses

A. A system for monitoring blood glucose levels, he system comprising: one or more processors; memory accessible by the one or more processors; headwear comprising: one or more electrodes positioned to contact a portion of skin and collect from the portion of skin electrical signals propagated from at least one eye and corresponding to an electrooculogram or electroretinogram; one or more grounding electrodes positioned on a nosebridge of the eyeglasses; and one or more modules maintained in the memory, which when executed by the one or more processors: receive the electrical signals collected by the one or more electrodes; amplify the electrical signals to remove noise from the electrical signals; filter the electrical signals to remove electrical signals with a frequency above or below a predetermined threshold; correlating the electrical signals to a blood glucose level; and outputting a current blood glucose level based at least in part on the compared electrical signal to a baseline blood glucose level.

B. A wearable device for monitoring bioelectric signals, the device comprising: one or more processors; one or more electrodes; memory accessible by the one or more processors having modules stored thereon, the modules, when executed by the one or more processors, configure the one or more processors to: c fleet at least one signal from the one or more electrodes; isolate a bioelectric signal from the collected signal; and transmit the bioelectric signal.

C. The wearable device as paragraph B recites, wherein the at least one bioelectric signal comprises at least one of a signal corresponding to an electrocardiogram, an electroencephalogram, an electromyogram, an electooculogram, or an electroretinogram.

D. The wearable device as paragraph B or C recites, wherein the type of second signal corresponds to literal features of and/or information regarding the image.

E. The wearable device as any one of paragraphs B-D recites further comprising a camera.

The wearable device as any one of paragraphs B-E recites, wherein the modules stored on the memory that, when executed by the one or more processors, further configure the processors to receive a second signal from a camera indicating a visible light state or infrared state.

G. The wearable device as any one of paragraphs recites, wherein isolating the bioelectric signal at least in part includes using the second signal.

H. The wearable device as any one of paragraphs B-G recites, wherein the at least one signal from the one or more electrodes is collected from one or more eyes of a user and the second signal indicates a gaze direction of the user.

I. The wearable device as any one of paragraphs recites, wherein isolating the bioelectric signal includes removing signal frequencies above a first threshold and below a second threshold from the received bioelectric signal and removing noise from the bioelectric signal.

J. The wearable device as any one of paragraphs B-I recites, wherein the wearable device is configured to continuously receive the at least one signal from the one or more electrodes and continuously identify at least one bioelectric signal from the one or more electrodes while the wearable device is placed on a user.

K. The wearable device as any one of paragraphs B-J recites, wherein the modules stored on the memory that, when executed by the one or more processors, further configure the processors to transmit the bioelectric signal to an external location, the external location comprising an electronic device communicatively coupled to the device and the electronic device is configured to display the information corresponding to the bioelectric signal.

L. The wearable device as any one of paragraphs B-K recites further comprising a display and wherein the wearable device transmits the bioelectric signal to the display

M. The wearable device as any one of paragraphs B-L recites further comprising at least one viewing lens and wherein the modules stored on the memory that, when executed by the one or more processors, further configure the processors to display information associated with the bioelectric signal on the at least one viewing lens.

N. The wearable device as any one of paragraphs B-M recites, wherein the modules stored on the memory that, when executed by the one or more processors, further configure the processors to calculate a health metric based at least in part on the bioelectric signal.

O. The wearable device as any one of paragraphs B-N recites, wherein the modules stored on the memory that, when executed by the one or more processors, further configure the processors to activate the light source to stimulate a user's eye and wherein the device isolates the bioelectric signal from the signal based at least in part on activation of the light source.

P. The wearable device as any one of paragraphs B-O recites further comprising a light source.

Q. A method comprising: collecting a bioelectric signal using electrodes disposed on or near one or more: skin near an eye, skin of a nasal bridge, skin of a temple, skin around or behind an ear, lateral canthus of an eye, medial canthus of an eye, or a surface of the eye; isolating a frequency spectrum from the collected bioelectric signal; correlating the isolated bioelectric signal to a biological condition state by, least in part, comparing the isolated bioelectric signal to a baseline bioelectric signal of the biological condition to produce biological condition data; and outputting the biological condition data.

R. The method as paragraph Q recites, wherein the electrodes are integrated with wearable eyeglasses and the bioelectric signal comprises at least one of a signal propagated through an organism corresponding to an electrocardiogram, an electroencephalogram, an electromyogram, an electooculogram, or an electroretinogram collected from the skin near the eye.

S. The method as paragraph Q or R recites, wherein the collected bioelectric signal is an analog signal and the acts further comprising converting the isolated bioelectric signal to a digital signal.

T. The method as any one of paragraphs Q-S recites further comprising: storing the current level of the biological condition based at least in part on the compared bioelectric signal to a baseline of the biological condition.

U. The method as any one of paragraphs Q-T recites, wherein the biological condition comprises at least one of a blood glucose level, a heart rate, a blood ketone level, a blood alcohol content, a hydration level, a blood albumin level, or a blood electrolyte level.

V. A method comprising: receiving the electrical signals collected by the one or more electrodes; amplifying the electrical signals to remove noise from the electrical signals; filtering the electrical signals to remove electrical signals with a frequency above or below a predetermined threshold; correlating the electrical signals to a blood glucose level; and outputting a current blood glucose level based at least in part on the compared electrical signal to a baseline blood glucose level,

W. A method comprising: collecting at least one signal from the one or more electrodes; isolating a bioelectric signal from the collected signal; and transmitting the bioelectric signal.

X. The method as any one of paragraphs Q-W recites, wherein the method is implemented by instructions stored on computer-readable media.

Y. The method as any one of paragraphs Q-W recites, wherein the method is implemented by a system comprising: a sensor; one or more processors; and computer-readable media having stored thereon computer-executable instructions, the computer-executable instructions configuring the one or more processors to perform the method.

Z. A computer-readable media having thereon computer-executable instructions to, upon execution, configure a computer to perform a method as any of paragraphs recites.

AA. A system comprising: one or more processors; and computer-readable media having thereon computer-executable instructions, the computer-executable instructions to configure the one or more processors to perform a method as any of paragraphs recites.

AB. A system comprising: means for processing; means for storing; and means for performing any steps of a method as any of paragraphs Q-W recites.

### CONCLUSION

Although the subject matter has been described in language specific to structural features and/or methodological acts, it is to be understood that the subject matter defined in the appended claims is not necessarily limited to the specific features or acts described. Rather, the specific features and acts are disclosed as example forms of implementing the claims.

All of the methods and processes described above can be embodied in, and fully automated via, software code modules and/or computer-executable instructions executed by one or more computers or processors. The code modules and/or computer executable instructions can be stored in any type of computer-readable medium. Some or all of the methods can alternatively be embodied in specialized computer hardware.

Conditional language such as, among others, “can,” “could,” “may” or “might,” unless specifically stated otherwise, are understood within the context to present that certain examples include, while other examples do not include, certain features, elements and/or steps. Thus, such conditional language is not generally intended to imply that certain features, elements and/or steps are in any way required for one or more examples or that one or more examples necessarily include logic for deciding, with or without user input or prompting, whether certain features, elements and/or steps are included or are to be performed in any particular example.

Conjunctive language such as the phrase “at least one of X, Y or Z,” unless specifically stated otherwise, is to be understood to present that an item, term, etc. can he either X, Y, or Z, or any combination thereof. Unless explicitly described as singular, “a” means singular and plural.

Any routine descriptions, elements or blocks in the flow diagrams described herein and/or depicted in the attached figures should be understood as potentially representing modules, segments, or portions of code that include one or more computer-executable instructions for implementing specific logical functions or elements in the routine. Alternate implementations are included within the scope of the examples described herein in which elements or functions can be deleted, or executed out of order from that shown or discussed, including substantially synchronously or in reverse order, depending on the functionality involved as would be understood by those skilled in the art.

It should be emphasized that many variations and modifications can be made to the above-described examples, the elements of which are to be understood as being among other acceptable examples. All such modifications and variations are intended to be included herein within the scope of this disclosure and protected by the following claims.

What is claimed is:

1. A system for monitoring blood glucose levels, the system comprising:
one or more processors;
memory accessible by the one or more processors;
headwear comprising:
one or more electrodes positioned to contact a portion of skin and collect from the portion of skin electrical signals propagated from at least one eye and corresponding to an electrooculogram or electroretinogram;
one or more grounding electrodes positioned on a nosebridge of the eyeglasses; and
one or more modules maintained in the memory, which when executed by the one or more processors:
receive the electrical signals collected by the one or more electrodes;
amplify the electrical signals to remove noise from the electrical signals;
filter the electrical signals to remove electrical signals with a frequency above or below a predetermined threshold;
correlate the electrical signals to a blood glucose level; and
output a current blood glucose level based at least in part on the compared electrical signal to a baseline blood glucose level.

2. A wearable device for monitoring bioelectric signals, the device comprising:
one or more processors;
one or more electrodes;
memory accessible by the one or more processors having modules stored thereon, the modules, when executed by the one or more processors, configure the one or more processors to:
collect at least one signal from the one or more electrodes;
isolate a bioelectric signal from the collected signal; and
transmit the bioelectric signal.

3. The device as recited in claim 2, wherein the at least one bioelectric signal comprises at least one of a signal corresponding to an electrocardiogram, an electroencephalogram, an electromyogram, an electooculogram, or an electroretinogram.

4. The device as recited in claim 2, further comprising a camera.

5. The device as recited in claim 4, wherein the modules stored on the memory that, when executed by the one or more processors, further configure the processors to receive a second signal from the camera indicating a visible light state or infrared state.

6. The device as recited in claim 5, wherein isolating the bioelectric signal at least in part includes using the second signal.

7. The device as recited in claim 5, wherein the at least one signal from the one or more electrodes is collected from one or more eyes of a user and the second signal indicates a gaze direction of the user.

8. The device as recited in claim 2, wherein isolating the bioelectric signal includes removing signal frequencies above a first threshold and below a second threshold from the received bioelectric signal and removing noise from the bioelectric signal.

9. The device as recited in claim 2, wherein the wearable device is configured to continuously receive the at least one signal from the one or more electrodes and continuously identify at least one bioelectric signal from the one or more electrodes while the wearable device is placed on a user.

10. The device as recited in claim 2, wherein the modules stored on the memory that, when executed by the one or more processors, further configure the processors to transmit the bioelectric signal to an external location, the external location comprising an electronic device communicatively coupled to the device and the electronic device is configured to display the information corresponding to the bioelectric signal.

11. The device as recited in claim 2 further comprising a display and wherein the wearable device transmits the bioelectric signal to the display.

12. The device as recited in claim 11, further comprising at least one viewing lens and wherein the modules stored on the memory that, when executed by the one or more processors, further configure the processors to display information associated with the bioelectric signal on the at least one viewing lens.

13. The device as recited in claim 2, wherein the modules stored on the memory that, when executed by the one or more processors, further configure the processors to calculate a health metric based at least in part on the bioelectric signal.

14. The device as recited in claim 2, further comprising a light source.

15. The device as recited in claim 12, wherein the modules stored on the memory that, when executed by the one or more processors, further configure the processors to activate the light source to stimulate a user's eye and wherein the device isolates the bioelectric signal from the signal based at least in part on activation of the light source.

16. A method comprising:
collecting a bioelectric signal using electrodes disposed on or near one or more: skin near an eye, skin of a nasal bridge, skin of a temple, skin around or behind an ear, lateral canthus of an eye, medial canthus of an eye, or a surface of the eye;
isolating a frequency spectrum from the collected bioelectric signal;
correlating the isolated bioelectric signal to a biological condition state by, at least in part, comparing the isolated bioelectric signal to a baseline bioelectric signal of the biological condition to produce biological condition data; and
outputting the biological condition data.

17. The method as recited in claim 16, wherein the electrodes are integrated with wearable eyeglasses and the bioelectric signal comprises at least one of a signal propagated through an organism corresponding to an electrocardiogram, an electroencephalogram, an electromyogram, an electooculogram, or an electroretinogram collected from the skin near the eye.

18. The method as recited in claim 16, wherein the collected bioelectric signal is an analog signal and the acts further comprising converting the isolated bioelectric signal to a digital signal.

19. The method as recited in claim 16, further comprising storing the current level of the biological condition based at least in part on the compared bioelectric signal to a baseline of the biological condition.

20. The method as recited in claim 16, wherein the biological condition comprises at least one of a blood glucose level, a heart rate, a blood ketone level, a blood alcohol content, a hydration level, a blood albumin level, or a blood electrolyte level.
